(19) United States
(12) Patent Application Publication
(10) Pub. No.: US 2022/0275078 A1
(43) Pub. Date: Sep. 1, 2022

(54) Treatment of Genital Psoriasis

(71) Applicant: Eli Lilly and Company, Indianapolis, IN (US)

(72) Inventors: Mark Oren Blakely, Indianapolis, IN (US); Alison Jean Potts Bleakman, New York, NY (US); Chin Hyok Lee, Indianapolis, IN (US); Brian Edward Wagner, Zionsville, IN (US)

(21) Appl. No.: 17/743,864

(22) Filed: May 13, 2022

Related U.S. Application Data
(63) Continuation of application No. 16/740,747, filed on Jan. 13, 2020.
Application No. 16/740,747 is a continuation of application No. PCT/US2018/044080, filed on Jul. 27, 2018.
(60) Provisional application No. 62/549,321, filed on Aug. 23, 2017.
(60) Provisional application No. 62/552,192, filed on Aug. 30, 2017.
(60) Provisional application No. 62/555,364, filed on Sep. 7, 2017.

(51) Int. Cl. C07K 16/24; A61P 15/00

(52) U.S. Cl. CPC C07K 16/244; A61P 15/00; A61K 38/00

(57) ABSTRACT

Methods of treating genital psoriasis, genital pruritus and genital psoriasis sexual activity impairment with anti-IL17A antibodies, including dose regimens and pharmaceutical formulations of anti-IL17A antibodies for use in the treatment of genital psoriasis, genital pruritus and genital psoriasis sexual activity impairment.

The present invention relates to the field of medicine. More particularly, the present invention relates to the use of anti-ILA antibodies for treatment of genital psoriasis (GenPs), genital pruritus, and genital psoriasis sexual activity impairment. The present invention also provides dose regimens and pharmaceutical formulations useful in the treatment of GenPs, genital pruritus, and genital psoriasis sexual activity impairment.

Psoriasis represents various immune-mediated skin diseases that involve multiple cell types and cytokines. Different types of psoriasis include plaque psoriasis, guttate, inverse, pustular, and erythrodermic, each of which present with varying forms of skin inflammation. Different psoriasis types often impact different patient populations and distinct regions of the body. Additionally, different types of psoriasis are known to have differing attendant comorbidities including obesity, cardiovascular disease, non-alcoholic fatty liver disease, diabetes, joint disease, cancer, depression as well as other autoimmune diseases. Further, different treatments are often necessary for different types of psoriasis, in-part due to variances in the skin at different regions of the body. Severity of psoriasis plays a primary role in dictating treatment options as well; and degree of severity can vary, even significantly, with different types of psoriasis. Severity of psoriasis, in general, is determined based on the percent of body surface area (“BSA”) impacted by a form of psoriasis. For example, some treatment requirements call for greater-than-or-equal-to 10% BSA to be impacted with a form of psoriasis before use of systemic treatments such as biologics.

Genital psoriasis (GenPs) impacts the skin of male and female genitalia (e.g., the labia majora, labia minora, and perineum for females, and the penis, scrotum, and perineum for males) and presentation of GenPs may vary between male and female genitalia. For example, GenPs of the vulva region may present as smooth, non-scaly bright red lesions whereas GenPs of the penile region may appear as small red patches on the glans or corona. GenPs may also include painful fissures and erosions as well as significant excoriations and lichenification, but often lacks the characteristic scale present at other body regions impacted by psoriasis. Furthermore, genital pruritus, a medical condition described as chronic and intense itching of genitalia, is reported in over 80% of GenPs patients and can have significant negative impact on a patient's quality of life.

GenPs has also been associated with greater stigmatization and lower self-esteem than other forms of psoriasis impacting other regions of body, including the face and hands, regardless of percent BSA impacted. Recent studies report that, independent of overall disease severity, when compared to non-GenPs psoriasis patients, patients with GenPs had a greater impairment of quality of life as measured by the overall Dermatology Life Quality Index (DLQI), Center for Epidemiological Studies Depression Scale (CES-D), and Relationship and Sexuality Scale (RLSS).

In addition to the painful skin manifestations, GenPs negatively impacts activities of daily living including a patient's sexual activity as defined by function (e.g., dyspareunia, mechanical friction, cracking, pain, and psychosocial effects including embarrassment and stigmatization impacting function and ability) and frequency (e.g., GenPs limiting patient engagement in sexual activity such as intercourse and masturbation) (the impact on sexual activity is referred to herein as “genital psoriasis sexual activity impairment”). Additionally, it has been reported that genital psoriasis symptoms friction, cracking, pain and burning may worsen during and/or following sexual activity (which, as used herein, refers to intercourse and masturbation).

Approximately 2-5% of patients with psoriasis present with psoriatic lesions only in the genital areas; however, several factors complicate treatment of GenPs. For example, the presentation of GenPs may lead to misdiagnosis including ailments such as dermatitis, tinea or candidiasis, squamous cell carcinoma, Zoon's plasma cell balanitis or vulvitis, lichen planus, secondary or tertiary (pustular) syphilis, balanitis circinata, lichen simplex chronicus (excoriated), extramammary Paget's disease, intertrigo, fixed drug eruption, lichen nitidus or sclerosus, and scabies or pediculosis pubis. Additionally, despite the significant impact on quality of life and, possibly as a result of social stigma, patients report not routinely discussing genital psoriasis with a health care professional. Another challenge complicating treatment of GenPs is that skin of the male and female genitalia is highly sensitive and at an increased risk of adverse reactions with typically recommended treatments such as topical therapies (including corticosteroids, calcineurin inhibitors, and vitamin D analogues) for psoriasis classified as mild in severity. Further, other typically recommended treatments for psoriasis classified as mild in severity, such as anthralin, tazarotene, ultraviolet light, and laser therapy are not appropriate for use in the genital area. Systemic therapy, such as biological treatments, are generally reserved for more extensive psoriasis cases whereas the genital region represents only approximately 1% of the body-surface area, meaning many patients with GenPs may not meet requisite BSA requirements for systemic treatment.

For at least the reasons provided herein, there exists an unmet need for an improved treatment of GenPs, genital pruritus and genital psoriasis sexual activity impairment. Such treatment should be appropriate for the male and female genitalia and provide therapeutic and daily living activity benefits for GenPs and genital pruritus patients. Additionally, such treatment should not be attendant on increased sexual dysfunction, but should improve or alleviate (in all or part) genital psoriasis sexual activity impairment. As with all therapeutic treatments, safety and toxicity remain a limitation, thus any improved treatments must not be attendant on unacceptable safety and toxicity profiles. The present invention provides a therapeutic treatment for the treatment of GenPs, genital pruritus and/or genital psoriasis sexual activity impairment which overcomes one or more of the challenges in treating GenPs, genital pruritus and/or genital psoriasis sexual activity impairment recognized above.

Accordingly, the present invention provides a treatment for genital psoriasis, genital pruritus and genital psoriasis sexual activity impairment which addresses one or more of the challenges described herein. In some embodiments, a method of treating a patient in need of treatment of one of genital psoriasis (GenPs), genital pruritus, and genital psoriasis sexual activity impairment is provided comprising administering to said patient a therapeutically effective amount of an anti-IL17A antibody, or a pharmaceutical formulation thereof. In more specific embodiments, the anti-IL17A antibody comprises a light chain variable region (LCVR) and a heavy chain variable region (HCVR), wherein said LCVR is the amino acid sequence of SEQ ID NO: 2 and said HCVR is the amino acid sequence of SEQ ID NO: 3. In even more specific embodiments, the anti-IL17A antibody comprises a light chain (LC) and a heavy chain (HC), wherein said LC is the amino acid sequence of SEQ ID NO: 4 and said HC is the amino acid sequence of SEQ ID NO: 5. In even more specific embodiments, the anti-IL17A antibody is ixekizumab.

According to some embodiments of the methods of treating provided herein, the patients possess a static Physician's Global Assessment of Genitalia (sPGA-G) of greater than sPGA-G 1. In other embodiments the patient possesses sPGA-G of greater-than-or-equal-to sPGA-G 3. In some embodiments, the patient does not need treatment for psoriasis at other body regions. In even further embodiments, the patient has less than 10% body-surface area (BSA) psoriasis involvement. In some embodiments, the patient has less-than-or-equal to 3% BSA psoriasis involvement. In even further embodiments, the patient has at least one of genital fissure and genital erosion.

According to some embodiments of the methods of treating provided herein, a pharmaceutical formulation of an anti-IL17A antibody is administered, the pharmaceutical formulation comprising an anti-IL17A antibody at a concentration of about 80 mg/mL, citrate buffer at a concentration of about 20 mM, sodium chloride at a concentration of about 200 mM, polysorbate-80 at a concentration in the range of about 0.02% (w/v) to about 0.03% (w/v), and pH at about 5.7. In even more specific embodiments, the concentration of polysorbate-80 is about 0.03% (w/v).

According to some of the methods of treatment provided by the present invention, administering comprises: (a) during a 12-week induction period, subcutaneously administering an initial dose of about 160 mg of the anti-IL17A antibody or pharmaceutical formulation thereof on Day 0, followed by administering subcutaneously about 80 mg of the anti-IL17A antibody or pharmaceutical formulation thereof at each of Weeks 2, 4, 6, 8, 10 and 12; and (b) following the 12-week induction period, administering subcutaneously about 80 mg of the anti-IL17A antibody or pharmaceutical formulation thereof at Week 16 and at every four week interval thereafter. In some embodiments, administering the initial dose of about 160 mg comprises administering two subcutaneous doses of about 80 mg of the anti-IL17A antibody or pharmaceutical formulation thereof on Day 0.

According to other embodiments of the methods of treatment provided by the present invention, administering comprises: administering subcutaneously an initial dose of about 160 mg of an anti-IL17A antibody or pharmaceutical formulation thereof on Day 0; and administering subcutaneously a dose of about 80 mg of the anti-IL17A antibody or pharmaceutical formulation thereof at every two-week interval following the initial dose. In some embodiments administering the initial dose of about 160 mg comprises administering two subcutaneous doses of about 80 mg of the anti-IL17A antibody or pharmaceutical formulation thereof on Day 0.

According to even further embodiments of the methods of treatment provided by the present invention, administering comprises: (a) during a 12-week induction period, administering subcutaneously an initial dose of about 160 mg of the anti-IL17A antibody or pharmaceutical formulation thereof on Day 0, followed by administering subcutaneous about 80 mg of the anti-IL17A antibody or pharmaceutical formulation thereof at each of Weeks 2, 4, 6, 8, 10 and 12; and (b) following the initial 12-week period, assessing the patient for sufficient clinical response and for patients assessed as not achieving (or maintaining) sufficient clinical response, administering subcutaneously about 80 mg of the anti-IL17A antibody or pharmaceutical formulation thereof every two weeks. In even more specific embodiments, administering the initial dose of about 160 mg comprises administering two subcutaneous doses of about 80 mg of the anti-IL17A antibody or pharmaceutical formulation thereof on Day 0. Further, in some embodiments, sufficient clinical response is one of: sPGA-G 0,1; sPGA-G 0; PatGA-G; or mGPASI. In some embodiments, the patient is assessed for sufficient clinical response at Week 12. In some embodiments, the patient is assessed for sufficient clinical response at Week 16. In even other embodiments, the patient is assessed for sufficient clinical response after Week 16. Even further embodiments, the patient is assessed at Week 40 and/or Week 52.

Furthermore, the present invention provides an anti-IL17A antibody or pharmaceutical formulation thereof, for use in the manufacture of a medicament for the treatment of GenPs, genital psoriasis sexual activity impairment, and/or genital pruritus. The present invention also provides an anti-IL17A antibody, or a pharmaceutical formulation thereof, for use in the treatment of one of GenPs, genital psoriasis sexual activity impairment, and genital pruritus, wherein a therapeutically effective amount of the anti-IL17A antibody or a pharmaceutical formulation thereof is administered to a patient in need of treatment for one of GenPs, genital psoriasis sexual activity impairment, and genital pruritus.

As referred to herein, the terms “individual,” “subject,” and “patient,” used interchangeably herein, refer to a human. In a certain embodiment, the subject is further characterized with a disease, disorder, or condition that would benefit from a decreased bioactivity of IL-17.

As used interchangeably herein, “treatment” and/or “treating” and/or “treat” are intended to refer to all processes wherein there may be a slowing, interrupting, arresting, controlling, stopping, or reversing of the progression of the disorders described herein, but does not necessarily indicate a total elimination of all disorder symptoms. Treatment includes administration of an antibody of the present invention for treatment of a disease or condition in a human that would benefit from a reduction in IL-17 activity, and includes: (a) inhibiting further progression of the disease, i.e., arresting its development; and (b) relieving the disease, i.e., causing regression of the disease or disorder or alleviating symptoms or complications thereof.

### Anti-IL17A Antibodies

As used interchangeably herein, anti-IL17 and anti-IL17A antibodies of the present invention refers to monoclonal antibodies of the present invention that specifically bind and antagonize human IL-17A with high affinity. As is known in the art, interleukin 17A, human IL17A, is a 20-30 kD dimeric (i.e., IL17AA homodimeric or IL17AF heterodimeric) glycoprotein which functions as a proinflammatory cytokine. According to specific embodiments, the anti-IL17A antibody comprises two light chain variable regions (LCVR) each having the amino acid sequence SEQ ID NO: 2 and two heavy chain variable regions (HCVR) each having the amino acid sequence SEQ ID NO: 3. In more specific embodiments, the anti-IL17A antibody comprises two light chains (LC) each having the amino acid sequence SEQ ID NO: 4 and two heavy chains (HC) each having the amino acid sequence SEQ ID NO: 5, and wherein the HCs are cross-linked by disulfide bonds. In preferred embodiments of the present invention the anti-IL17A antibody is ixekizumab. Further characterization of antibodies for use in the present invention are provided in U.S. Pat. No. 7,838,638.

### Pharmaceutical Formulation of Anti-IL17A Antibodies

Embodiments of the present invention also include the use of anti-IL17A antibodies incorporated in pharmaceutical formulations. A pharmaceutical formulation, as used herein, is a stable formulation comprising the anti-IL17A antibody of the present invention, preferably ixekizumab, wherein the degree of degradation, modification, aggregation, loss of biological activity and the like, of proteins/antibodies therein is acceptably controlled, and does not increase unacceptably with time. Stability of an antibody in solution depends on the chemical and physical stability of the antibody in the formulation in which the antibody is solubilized. Issues such as oxidation, deamidation, and hydrolysis are examples of chemical stability issues while aggregation, gel formation and liquid-liquid phase separation are examples of physical stability issues that an antibody can have in a formulation. Stability may be assessed by methods well-known in the art, including measurement of a sample's light scattering, apparent attenuation of light (absorbance, or optical density), size (e.g. by size exclusion chromatography (SEC)), in vitro or in vivo biological activity and/or properties measured by differential scanning calorimetry (DSC).

Pharmaceutical formulations for use in the present invention can be in the liquid dosage form of a solution, emulsion, or suspension and may be administered via parenteral administration including intravenous, intramuscular, subcutaneous, and intraperitoneal. Specific embodiments of anti-IL17A antibody pharmaceutical formulations of the present invention include the pharmaceutical formulation of Table 1.

TABLE 1
Anti-IL-17A Antibody Pharmaceutical Formulation

| | Concentration |
|---|---|
| Component | (mg/mL) |
| Anti-IL-17 antibody | 80 |
| Sodium Citrate Dihydrate | 5.106 |
| Citric Acid Anhydrous | 0.507 |
| Sodium Chloride | 11.69 |
| Polysorbate 80 | 0.30 |
| Water for Injection | q.s. to 1 mL |
| Hydrochloric Acid | pH adjustment |
| Sodium Hydroxide | pH adjustment |

Further characterization of anti-IL17A antibody pharmaceutical formulations for use in the present invention are provided in U.S. Pat. No. 9,376,491. Preferred embodiments of the pharmaceutical formulations for use in the present invention include the anti-IL17A antibody being ixekizumab, and even more preferably include the commercially available pharmaceutical formulation Taltz®.

### Dose Regimen

The present invention also relates to dose regimens for the treatment of GenPs, genital pruritus, and genital psoriasis sexual activity impairment with anti-IL17A antibodies. As referred to herein and as generally known in the art, the term “dose” refers to an amount (e.g., a concentration) of anti-IL17A antibody that is administered to a subject. A “dose regimen” or “dosage regimen” as generally known in the field and as may be referred to interchangeably herein includes a treatment schedule for administering a set (i.e., series or sequence) of doses to be administered to a patient over a period of time.

In an exemplary embodiment of a dose regimen provided with the GenPs, genital pruritus, and genital psoriasis sexual activity impairment treatments of the present invention, during an initial 12-week (“induction”) period, an initial dose of about 160 mg of an anti-IL17A antibody or pharmaceutical formulation thereof (comprising two separate doses of about 80 mg) is administered subcutaneously on the first day of treatment (e.g., Day 0), followed by subcutaneous administration of treatment doses of about 80 mg of the anti-IL17A antibody or formulation thereof at 14-day (or two week intervals) (e.g., a treatment dose given at each of Days 14, 28, 42, 56, 70 and 84 during the initial 12-week period). According to such embodiments, following the initial 12-week period, the anti-IL17A antibody or pharmaceutical formulation thereof is subcutaneously administered in treatment doses of about 80 mg of the anti-IL17A antibody or formulation thereof at 28-day (or four week) intervals (e.g., a treatment dose given at each of Days 112, 140, 168, 196 and so on) during the period, sometimes referred to as the “maintenance period,” which follows the initial 12-week period.

Another exemplary dose regimen includes administration of anti-IL17A antibody, or pharmaceutical formulation thereof, wherein the antibody or formulation thereof, at a given dose (e.g., two separate 80 mg subcutaneous injections) is first administered as an initial dose on a first day (e.g., Day 0) and then administered in treatment doses (e.g., a single 80 mg subcutaneous injection) given at every two week interval (e.g., Day 14, 28, 42, 56, 70, 84 and so on respectively).

In another exemplary embodiment of a dose regimen provided herein, during an initial 12-week induction period, an initial dose of about 160 mg of an anti-IL17A antibody or pharmaceutical formulation thereof (comprising two separate doses of about 80 mg) is administered subcutaneously on the first day of treatment (e.g., Day 0), followed by subcutaneous administration of treatment doses of about 80 mg of the anti-IL17A antibody or formulation thereof at 14-day (e.g., two week intervals) (for example, a treatment dose given at each of Days 14, 28, 42, 56, 70 and 84 during the initial 12-week induction period), whereby following the initial 12-week period, the patient is assessed for a desired or sufficient clinical response. A desired or sufficient clinical response, for example, with GenPs, may be based on sPGA-G 0,1; sPGA-G 0; PatGA-G; or mGPASI or the like, or a combination of any of these assessment metrics. Patients not achieving (or maintaining) the desired or sufficient clinical response (e.g., sPGA-G 0,1; sPGA-G 0; PatGA-G; or mGPASI) are subcutaneously administered about 80 mg of the anti-IL17A antibody or pharmaceutical formulation thereof every two weeks for an additional period of time (for example, for an additional 16 weeks after the initial 12-week period). According to such embodiments, the patient may be assessed for (achieving or maintaining) a desired or sufficient clinical response at Week 12, or any week following the initial 12-week period.

Also, as provided herein, the instant invention provides improved methods of treating, including improved dose regimens, for the treatment of GenPs, genital pruritus and genital psoriasis sexual activity impairment, such improved methods of treating and dose regimens comprising administering anti-IL17A antibodies (or pharmaceutical formulation thereof) to a patient in need of treatment for one or more of GenPs, genital pruritus and genital psoriasis sexual activity impairment. In preferred embodiments of improved methods and dose regimens provided herein, the anti-IL17A antibody is ixekizumab.

Further, as should be understood, Day 0 as used herein refers to the day on which the initial dose is administered (which, in at least some dose regimens provided herein marks the start of an “initial 12-week period”). Also, as referred to herein, “Day 14” or “Day 14, 28,” and so on, refers to the calendar day which comes 14 days (or 28 days in the case of Day 28; or 42 days in the case of Day 42, and so on) after Day 0, inclusive of Day 14, 28 and so on. By way of example, if Day 0 falls on the calendar date January 1^{st}, Day 14 would fall on the calendar date January 15^{th} and Day 28 would fall on the calendar date January 29^{th} and so on. Likewise, when referred to herein, “2 week interval “or “2 week intervals” refer to the calendar days which come on the 14 day intervals following Day 0 (e.g., administration of the initial dose).

sPGA

As referred to herein, “static Physician Global Assessment” and “sPGA”, as used interchangeably herein, refers to a physician's global assessment of a patient's psoriasis lesions at a given point. As understood in the art, the sPGA includes assessment of the psoriasis lesions for induration, erythema, and scaling, and an overall rating of psoriasis severity is given using the anchors of clear (0), minimal (1), mild (2), moderate (3), severe (4), or very severe (5). By way of example, an assessment of sPGA 0,1, is understood to represent clinically meaningful response of minimal plaque severity and/or complete resolution of psoriasis plaque (where sPGA 0 is indicative of complete resolution of psoriatic plaques). Information, as understood in the art, regarding sPGA is further provided at “European Medicines Agency, Committee for Medicinal Products for Human Use, Guidelines on clinical investigation of medicinal products indicated for the treatment of psoriasis, November 2004, available at:

https://www.ema.europa.eu/en/documents/scientific-guideline/guideline-clinical-investigation-medicinal-products-indicated-treatment-psoriasis_en.pdf.

sPGA-G

As referred to herein, “static Physician Global Assessment of Genitalia” and “sPGA-G”, as used interchangeably herein, refer to a physician's global assessment of a patient's GenPs lesions at a given point. The assessment area includes the vulvar region from the clitoral prepuce to the perineum, including the labia majora, labia minora, and perineum for females, and the penis, scrotum, and perineum for males. As understood in the art, sPGA-G includes assessment of GenPs lesions for erythema, induration and scaling (as may be present), and an overall rating of GenPs severity is given using the anchors of clear (0), minimal (1), mild (2), moderate (3), severe (4), or very severe (5). An assessment of sPGA-G 0,1, is understood to represent clinically meaningful response of minimal GenPs lesion severity and/or complete resolution of GenPs (where sPGA-G 0 is indicative of complete resolution of GenPs). Information, as understood in the art, regarding sPGA-G is further provided at Joseph E. Merola et al. (2016). The Static Physician's Global Assessment of Genitalia: A Clinical Outcome Measure for the Severity of Genital Psoriasis. Journal of Drugs and Dermatology, Volume 16, Issue 8, available at:

http://j ddonline.com/articles/dermatology/S1545961617P0793X/1.
mGPASI

As referred to herein, “modified Genital Psoriasis Area and Severity Index” and “mGPASI” as used interchangeably herein, refers to a physician's assessment of a patient's GenPs at a given point. As understood in the art, mGPASI includes assessment of labia majora, labia minora, and perineum (in females); penis, scrotum, and perineum (in males) yielding an overall score which incorporates the degree of erythema, induration, and scaling of GenPs plaques as well as erosion, fissure, and/or ulcer (where mGPASI 0 is understood to represent a meaningful response and/or no GenPs, and mGPASI 72 is indicative of the most severe GenPs). Information, as understood in the art, regarding mGPASI is further provided at: Bissonnette et al, 2008 (male genital PASI), available at https://www.ncbi.nlm.nih.gov/pubmed/18845092; and Ryan et al. 2016 (genital PASI) available at https://www.ncbi.nlm.nih.gov/pubmed/25824273.

### PatGA-G

As referred to herein, “Patient's Global Assessment of Genital Psoriasis” or “PatGA-G”, as used interchangeably herein, is a patient-administered, single-item scale on which patients rank, from 0 to 5, the severity of their GenPs at a given point. A rank, or value, of 0 represents clear or no GenPs; a value of 5 represents the most severe GenPs.

### EXAMPLES

A double-blind, parallel group clinical study comparing ixekizumab (an anti-IL17A antibody having a LC with the amino acid sequence of SEQ ID NO. 4 and a HC with the amino acid sequence of SEQ ID NO. 5, and further described in U.S. Pat. Nos. 7,838,638 and 9,376,491) to placebo in patients with genital psoriasis, genital psoriasis sexual activity impairment, or genital pruritus, as described herein, is conducted over a 52 week period (not inclusive of up to a 24 week post-treatment follow-up period). Prior to treatment, patients are screened for eligibility during a period of 7 to 30 days. Eligible patients are then randomized to one of an ixekizumab treatment group or placebo group. On Day 0, patients are subcutaneously administered an initial dose of 160 mg of either ixekizumab (administered as two subcutaneous 80 mg injections) or placebo, respectively, and then a treatment dose of 80 mg of either ixekizumab or placebo (administered subcutaneously), respectively, at every two week interval starting from Day 0 (e.g., at Week 2, Week 4, Week 6, Week 8, and Week 10). Safety and efficacy is assessed for respective patients groups at Week 12.

At Week 12, the ixekizumab treatment group receives a treatment dose of 80 mg of ixekizumab and the placebo group receives a treatment dose of 160 mg of ixekizumab (administered as two subcutaneous 80 mg injections). Following treatment at Week 12, both placebo and ixekizumab treatment groups, respectively, are subcutaneously administered a treatment dose of 80 mg of ixekizumab at every four week interval (e.g., Week 16, Week 20, Week 24, and so on), with a possible dose frequency adjustment to every two weeks starting at Week 24. Treatment for both placebo and ixekizumab treatment groups (including patients moved to a dose frequency adjustment of every two-week treatment) is continued through Week 52. Following Week 52, patients are monitored for safety issue signals during a post-treatment follow-up period of between twelve and twenty-four weeks.

No serious adverse events are reported for either treatment group; one serious adverse event is reported in the placebo treatment group.

### Genital Psoriasis

Ixekizumab impact on GenPs is assessed according to sPGA-G 0,1. As described above, ixekizumab is compared to placebo in a double-blind, parallel group clinical study. The percentage of patients achieving sPGA-G 0,1, in the ixekizumab treatment group (N=75) is compared to the percentage of patients achieving sPGA-G 0,1 in the placebo group (N=74). At Day 0, only a single patient in either treatment group is assessed as sPGA-G 0,1. Results through Week 12 are provided in Table 2.

TABLE 2
Percentage (%) of Patients Achieving sPGA-G 0,1

| Week | 1 | 2 | 4 | 8 | 12 |
|---|---|---|---|---|---|
| Placebo | 1.4 | 1.4 | 2.7 | 8.1 | 8.1 |
| (N = 74) | | | | | |
| Ixekizumab treatment group | 24.0 | 45.3 | 56.0 | 72.0 | 73.3 |
| (N = 75) | | | | | |

Ixekizumab impact on GenPs is assessed according to sPGA-G 0. As described above, ixekizumab is compared to placebo in a double-blind, parallel group clinical study. The percentage of patients achieving sPGA-G 0 in the ixekizumab treatment group (N=75) is compared to the percentage of patients achieving sPGA-G 0 in the placebo group (N=74). At Day 0, no patients in either group are assessed as sPGA-G 0. Results through Week 12 are provided in Table 3.

TABLE 3
Percentage (%) of Patients Achieving sPGA-G 0

| Week | 1 | 2 | 4 | 8 | 12 |
|---|---|---|---|---|---|
| Placebo | 0 | 0 | 0 | 5.4 | 5.4 |
| (N = 74) | | | | | |
| Ixekizumab treatment group | 8.0 | 21.3 | 30.7 | 53.3 | 56.0 |
| (N = 75) | | | | | |

Ixekizumab impact on GenPs in view of percent body surface area (“BSA”) involved or affected by psoriasis is assessed according to sPGA-G 0,1. As described above, ixekizumab is compared to placebo in a double-blind, parallel group clinical study. Patients in the ixekizumab treatment group are assessed and assigned to one of: less than 10% BSA psoriasis involved (N=31); or greater than or equal to 10% BSA psoriasis involved (N=44). Patients in the placebo group assessed as less than 10% BSA psoriasis involved (N=28) are also included. The percentage of patients achieving sPGA 0,1 is compared for the three treatment and % BSA psoriasis involved groups. At Day 0, only a single patient in either treatment group is assessed as sPGA-G 0, 1. Results following assessment at Week 12 are provided in Table 4.

TABLE 4
Percentage (%) of Patients Achieving sPGA-G 0,1

| Week | 12 |
|---|---|
| <10% BSA (Placebo) | 0 |
| (N = 28) | |
| <10% BSA (ixekizumab) | 71.0 |
| (N = 31) | |
| ≥10% BSA (placebo) | 13.0 |
| (N = 46) | |
| ≥10% BSA (ixekizumab) | 75.0 |
| (N = 44) | |

Ixekizumab impact on GenPs in view of percent body surface area (“BSA”) involved or affected by psoriasis is assessed according to sPGA-G 0. As described above, ixekizumab is compared to placebo in a double-blind, parallel group clinical study. Patients in the ixekizumab treatment group are assessed and assigned to one of: less than 10% BSA psoriasis involved (N=31); or greater than or equal to 10% BSA psoriasis involved (N=44). Patients in the placebo group assessed as less than or equal to 10% BSA psoriasis involved (N=28) are also included. The percentage of patients achieving sPGA 0 is compared for the three treatment and % BSA psoriasis involved groups. At Day 0, no patients are assessed as sPGA-G 0. Results following assessment at Week 12 are provided in Table 5.

TABLE 5
Percentage (%) of Patients Achieving sPGA-G 0

| Week | 12 |
|---|---|
| <10% BSA (placebo) | 0 |
| (N = 28) | |
| <10% BSA (ixekizumab) | 48.4 |
| (N = 31) | |
| ≥10% BSA (placebo) | 8.7 |
| (N = 46) | |
| ≥10% BSA (ixekizumab) | 61.4 |
| (N = 44) | |

Ixekizumab impact on GenPs is assessed according to mGPASI. As described above, ixekizumab is compared to placebo in a double-blind, parallel group clinical study. All GenPs patients are assessed at the start of the clinical study to establish a mGPASI baseline. Results for patients in the ixekizumab treatment group (N=74) are compared to results of patients in the placebo group (N=73) and results are provided according to least square mean (LSM) change from baseline. Results at Weeks: 1, 2, 4, 8 and 12 are provided in Table 6.

TABLE 6
mGPASI Assessment of LSM Change from Baseline

| Week | 1 | 2 | 4 | 8 | 12 |
|---|---|---|---|---|---|
| Placebo | −2.5 | −4.2 | −5.1 | −6.4 | −3.9 |
| (N = 73) | | | | | |
| Ixekizumab treatment group | −12.7 | −18.1 | −20.1 | −23.1 | −23.9 |
| (N = 74) | | | | | |

Ixekizumab impact on GenPs is assessed according to PatGA-G. As described above, ixekizumab is compared to placebo in a double-blind, parallel group clinical study. All GenPs patients are assessed at the start of the clinical study to establish a PatGA-G baseline. Results for patients in the ixekizumab treatment group (N=74) are compared to results of patients in the placebo group (N=73) and results are expressed as a percentage of patients assessed as having at least a two-point improvement from baseline (e.g., from a 5 to a 3 or a 4 to a 2). Results at Weeks: 1, 2, 4, 8 and 12 are provided in Table 7.

TABLE 7
Percentage (%) of Patients Having at
Least a Two-Point PatGA-G Improvement

| Week | 1 | 2 | 4 | 8 | 12 |
|---|---|---|---|---|---|
| Placebo | 8.2 | 12.3 | 13.7 | 17.8 | 15.1 |
| (N = 73) | | | | | |
| Ixekizumab treatment group | 36.1 | 41.7 | 62.5 | 70.8 | 70.8 |
| (N = 72) | | | | | |

### Genital Pruritus

Ixekizumab impact on genital pruritus is assessed according to the 11 point (0-10) patient reported Genital Psoriasis Symptom Scale (“GPSS”) assessment for itch in which 0 represents no itch and 10 represents the worst imaginable itch. As described above, ixekizumab is compared to placebo in a double-blind, parallel group clinical study. All genital pruritus patients are assessed at the start of the clinical study to establish a baseline. Patient assessment under the patient reported GPSS assessment for itch, is performed on a daily basis for the first twelve weeks of treatment. Following the initial twelve-week treatment period, assessment occurs only upon physician visit. Results for patients in the ixekizumab treatment group (N=62) are compared to results of patients in the placebo group (N=60) and results are expressed as a percentage of patients achieving a greater than or equal to 3 point improvement from baseline. At Week 12, 59.7% of patients in the ixekizumab treatment group (N=62) achieved a greater than or equal to improvement of at least 3 points from baseline (with an average score improvement of −4.02±0.27 (LSM±Standard Error)), compared to only 8.3% of patients in the placebo group (N=60) (with an average score improvement of −0.21±0.29 (LSM±Standard Error)).

### Genital Psoriasis Sexual Activity Impairment

Ixekizumab impact on sexual activity impairment is assessed according to patient reported sexual frequency assessment. As described above, ixekizumab is compared to placebo in a double-blind, parallel group clinical study. Patients are asked to provide a score of 0-4 (0 (never), 1 (rarely), 2 (sometimes), 3 (often), 4 (always)) for how often, during a past week, sexual activity (including intercourse and masturbation) was limited as a result of genital psoriasis. Patients provide a score of 1-5 during the 7 to 30 day pre-treatment period to establish a baseline. Results for patients in the ixekizumab treatment group (N=37) are compared to results of patients in the placebo group (N=42) and results are expressed as a percentage of patients achieving a greater than or equal to 2 point improvement from baseline. All patients have a baseline value of at least 2. At Week 12, 73% of patients in the ixekizumab treatment group achieved a greater than or equal to improvement of at least 2 points from baseline, compared to only 16.7% of patients in the placebo group. Results for patients in the ixekizumab treatment group are also compared to results of patients in the placebo group for percentage of patients achieving a score of 0 (never) or 1 (rarely) and are provided in Table 8.

TABLE 8
Percentage (%) of Patients Achieving a Sexual Frequency
Assessment Score of 0 (Never) or 1 (Rarely)

| Week | 2 | 4 | 8 | 12 |
|---|---|---|---|---|
| Placebo | 14.3 | 19.0 | 21.4 | 21.4% |
| (N = 42) | | | | |
| Ixekizumab | 32.4 | 51.4 | 67.6 | 78.5% |
| (N = 37) | | | | |

Ixekizumab impact on sexual activity impairment is assessed according to patient reported sexual impact assessment. As described above, ixekizumab is compared to placebo in a double-blind, parallel group clinical study. Sexually active patients are asked to provide a score of 1-5 (1 (never), 2 (rarely), 3 (sometimes), 4 (often), 5 (always)) for how often, during a past week, the patient experienced worsening of genital psoriasis symptoms (including friction, burning, and cracking) during and/or after sexual activity (including intercourse and masturbation). Patients provide a score of 1-5 during the 7 to 30 day pre-treatment period to establish a baseline. All patients have a baseline value of at least 3. Results for patients in the ixekizumab treatment group are compared to results of patients in the placebo group and results are expressed as a percentage of patients achieving a score of 1 (never) or 2 (rarely). Results for the first twelve-week treatment period are provided in Table 9.

TABLE 9
Percentage (%) of Patients Achieving a Sexual Impact
Assessment Score of 1 (never) or 2 (rarely)

| Week | 2 | 4 | 8 | 12 |
|---|---|---|---|---|
| Placebo | 25.0 | 27.8 | 44.4 | 52.9 |
| | (N = 20) | (N = 18) | (N = 18) | (N = 17) |
| Ixekizumab | 58.8 | 73.3 | 86.7 | 85.7 |
| | (N = 17) | (N = 15) | (N = 15) | (N = 14) |

Ixekizumab impact on sexual activity impairment is assessed according to a patient reported avoidance assessment. As described above, ixekizumab is compared to placebo in a double-blind, parallel group clinical study. Patients are asked to provide a score of 1-5 (1 (never), 2 (rarely), 3 (sometimes), 4 (often), 5 (always)) for how often, during a past week, the patient avoided intercourse and/or masturbation as a result of genital psoriasis. Patients provide a score of 1-5 during the 7 to 30 day pre-treatment period to establish a baseline. Results for patients in the ixekizumab treatment group (N=30) are compared to results of patients in the placebo group (N=35) and results are expressed as a percentage of patients achieving a score of 1 (never) or 2 (rarely). All patients have a baseline value of at least 3. At Week 12, approx. 77% of patients in the ixekizumab treatment group achieved a score of 1 (never) or 2 (rarely), compared to only approx. 26% of patients in the placebo group. Results are provided in Table 10.

TABLE 10
Percentage (%) of Patients Achieving an Avoidance
Score of 1 (Never) or 2 (Rarely)

| Week | 2 | 4 | 8 | 12 |
|---|---|---|---|---|
| Placebo | 17.1 | 22.9 | 22.9 | 25.7% |
| (N = 35) | | | | |
| Ixekizumab | 30.0 | 63.3 | 63.3 | 76.7% |
| (N = 30) | | | | |

Sequence Listing
(human IL-17)
SEQ ID NO: 1
MTPGKTSLVS LLLLLSLEAI VKAGITIPRN
PGCPNSEDKN FPRTVMVNLN IHNRNTNTNP
KRSSDYYNRS TSPWNLHRNE DPERYPSVIW
EAKCRHLGCI NADGNVDYHM NSVPIQQEIL
VLRREPPHCP NSFRLEKILV SVGCTCVTPI
VHHVA
(LCVR)
SEQ ID NO: 2
DIVMTQTPLS LSVTPGQPAS ISCRSSRSLV
HSRGNTYLHW YLQKPGQSPQ LLIYKVSNRF
IGVPDRFSGS GSGTDFTLKI SRVEAEDVGV
YYCSQSTHLP FTFGQGTKLE IK
(HCVR)
SEQ ID NO: 3
QVQLVQSGAE VKKPGSSVKV SCKASGYSFT
DYHIHWVRQA PGQGLEWMGV INPMYGTTDY
NQRFKGRVTI TADESTSTAY MELSSLRSED
TAVYYCARYD YFTGTGVYWG QGTLVTVSS
(Light chain)
SEQ ID NO: 4
DIVMTQTPLS LSVTPGQPAS ISCRSSRSLV
HSRGNTYLHW YLQKPGQSPQ LLIYKVSNRF
IGVPDRFSGS GSGTDFTLKI SRVEAEDVGV
YYCSQSTHLP FTFGQGTKLE IKRTVAAPSV
FIFPPSDEQL KSGTASVVCL LNNFYPREAK
VQWKVDNALQ SGNSQESVTE QDSKDSTYSL
SSTLTLSKAD YEKHKVYACE VTHQGLSSPV
TKSFNRGEC
(Heavy chain)
SEQ ID NO: 5
QVQLVQSGAE VKKPGSSVKV SCKASGYSFT
DYHIHWVRQA PGQGLEWMGV INPMYGTTDY
NQRFKGRVTI TADESTSTAY MELSSLRSED
TAVYYCARYD YFTGTGVYWG QGTLVTVSSA
STKGPSVFPL APCSRSTSES TAALGCLVKD
YFPEPVTVSW NSGALTSGVH TFPAVLQSSG
LYSLSSVVTV PSSSLGTKTY TCNVDHKPSN
TKVDKRVESK YGPPCPPCPA PEFLGGPSVF
LFPPKPKDTL MISRTPEVTC VVVDVSQEDP
EVQFNWYVDG VEVHNAKTKP REEQFNSTYR
VVSVLTVLHQ DWLNGKEYKC KVSNKGLPSS
IEKTISKAKG QPREPQVYTL PPSQEEMTKN
QVSLTCLVKG FYPSDIAVEW ESNGQPENNY
KTTPPVLDSD GSFFLYSRLT VDKSRWQEGN
VFSCSVMHEA LHNHYTQKSL SLSLG

We claim:

1. A method of treating a patient having less than 10% body surface area psoriasis involvement, comprising administering to the patient a therapeutically effective amount of an anti-IL17A antibody, or a pharmaceutical formulation thereof,
wherein the anti-IL17A antibody comprises a light chain variable region (LCVR) and a heavy chain variable region (HCVR), wherein said LCVR is the amino acid sequence of SEQ ID NO: 2 and said HCVR is the amino acid sequence of SEQ ID NO: 3.

2. The method of claim 1, wherein the anti-IL17A antibody comprises a light chain (LC) and a heavy chain (HC), wherein the LC is the amino acid sequence of SEQ ID NO: 4 and the HC is the amino acid sequence of SEQ ID NO: 5.

3. The method of claim 2, wherein the anti-IL17A antibody is ixekizumab.

4. The method of claim 1, wherein the patient has greater than 3% body surface area psoriasis involvement.

5. The method of claim 1, wherein the patient has less-than 3% body surface area psoriasis involvement.

6. The method of claim 1, wherein said step of administering comprises
(a) during a 12-week induction period, administering an initial dose subcutaneous of about 160 mg of the anti-IL17 antibody or pharmaceutical formulation thereof on day 0, followed by administering subcutaneous about 80 mg of the anti-IL17 antibody or pharmaceutical formulation thereof at each of weeks 2, 4, 6, 8, 10 and 12; and
(b) following the 12-week induction period, administering subcutaneous about 80 mg of the anti-IL17 antibody or pharmaceutical formulation thereof at week 16 and at every four-week interval thereafter.

7. The method of claim 1, wherein said step of administering comprises, during a 12-week induction period administering an initial dose subcutaneous of about 160 mg of the anti-IL17 antibody or pharmaceutical formulation thereof on day 0, followed by administering subcutaneous about 80 mg of the anti-IL17 antibody or pharmaceutical formulation thereof at every two-week interval thereafter.

8. A method of achieving one of static physician's global assessment of genitalia 0, static physician's global assessment of genitalia 1 or static physician global assessment 0 in a patient having genital psoriasis, comprising administering to the patient a therapeutically effective amount of an anti-IL17A antibody, or a pharmaceutical formulation thereof,
wherein the anti-IL17A antibody comprises a light chain variable region (LCVR) and a heavy chain variable region (HCVR), wherein the LCVR is the amino acid sequence of SEQ ID NO: 2 and the HCVR is the amino acid sequence of SEQ ID NO: 3,
wherein following said step of administering, the patient achieves at least one of static physician's global assessment of genitalia 0, static physician's global assessment of genitalia 1 or static physician global assessment 0.

9. The method of claim 8, wherein the anti-IL17A antibody comprises a light chain (LC) and a heavy chain (HC), wherein the LC is the amino acid sequence of SEQ ID NO: 4 and the HC is the amino acid sequence of SEQ ID NO: 5.

10. The method of claim 9, wherein the anti-IL17A antibody is ixekizumab.

11. The method of claim 8, wherein the patient has less than 10% body surface area psoriasis involvement.

12. The method of claim 11, wherein the patient has greater than 3% body surface area psoriasis involvement.

13. The method of claim 8, wherein the patient has less-than 3% body surface area psoriasis involvement.

14. The method of claim 8, wherein said step of administering comprises
(a) during a 12-week induction period, administering an initial dose subcutaneous of about 160 mg of the anti-IL17 antibody or pharmaceutical formulation thereof on day 0, followed by administering subcutaneous about 80 mg of the anti-IL17 antibody or pharmaceutical formulation thereof at each of weeks 2, 4, 6, 8, 10 and 12; and
(b) following the 12-week induction period, administering subcutaneous about 80 mg of the anti-IL17 antibody or pharmaceutical formulation thereof at week 16 and at every four-week interval thereafter until week 52.

15. The method of claim 8, wherein said step of administering comprises, during a 12-week induction period administering an initial dose subcutaneous of about 160 mg of the anti-IL17 antibody or pharmaceutical formulation thereof on day 0, followed by administering subcutaneous about 80 mg of the anti-IL17 antibody or pharmaceutical formulation thereof at every two-week interval thereafter until week 52.

16. The method of claim 8, wherein the patient has at least one of genital fissure and genital erosion.

17. The method of claim 8, wherein the patient does not have psoriasis at body regions other than the patient's genital region.

18. The method of claim 8, wherein following said step of administering the patient achieves static physician's global assessment of genitalia 1.

19. The method of claim 8, wherein following said step of administering the patient achieves static physician's global assessment of genitalia 0.

20. A method of achieving static physician's global assessment of genitalia 1 in a patient having genital psoriasis and having at least one of genital fissure and genital erosion and having less than 10% and greater than 1% body surface area psoriasis involvement, comprising administering to said patient a therapeutically effective amount of an anti-IL17A antibody, or a pharmaceutical formulation thereof, wherein the anti-IL17A antibody comprises a light chain (LC) and a heavy chain (HC), wherein the LC is the amino acid sequence of SEQ ID NO: 4 and the HC is the amino acid sequence of SEQ ID NO: 5,
wherein said step of administering comprises (a) during a 12-week induction period, administering an initial dose subcutaneous of about 160 mg of the anti-IL17 antibody or pharmaceutical formulation thereof on day 0, followed by administering subcutaneous about 80 mg of the anti-IL17 antibody or pharmaceutical formulation thereof at each of weeks 2, 4, 6, 8, 10 and 12; and (b) following the 12-week induction period, administering subcutaneous about 80 mg of the anti-IL17 antibody or pharmaceutical formulation thereof at week 16 and at every four-week interval thereafter until week 52, and
wherein following said step of administering the patient achieves static physician's global assessment of genitalia 1.


SEQUENCE LISTING (5 sequences)

SEQ ID NO: 1 - protein, 155 aa, Artificial Sequence
  hIL-17
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 2 - protein, 112 aa, Artificial Sequence
  LCVR
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIK

SEQ ID NO: 3 - protein, 119 aa, Artificial Sequence
  HCVR
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSS

SEQ ID NO: 4 - protein, 219 aa, Artificial Sequence
  Light chain
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 445 aa, Artificial Sequence
  Heavy chain
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG